(19) United States
(12) Patent Application Publication
(10) Pub. No.: US 2006/0247519 A1
(43) Pub. Date: Nov. 2, 2006

(54) Contrast medium infusion method and lesion imaging system

(76) Inventor: Yasushi Kawamoto, Kyoto-City (JP)

Correspondence Address: GREGORY L BRADLEY;MEDRAD INC, ONE MEDRAD DRIVE, INDIANOLA, PA, 15051, US

(21) Appl. No.: 11/389,689

(22) Filed: Mar. 27, 2006

(30) Foreign Application Priority Data
Nov. 17, 2004 (JP) 2004-332757

(51) Int. Cl. A61M 5/00

(52) U.S. Cl. 600/432

(57) ABSTRACT

The invention provides a method of infusing contrast medium that includes a process of infusing a small amount of contrast medium from a syringe and using an image capturing device to measure a time Tarv from a start of infusion until contrast medium arrives at a lesion area, a process of subtracting the arrival time Tarv from a time Tscan necessary for infusion of a sufficient amount of contrast medium and taking this as a time T1, a process of starting infusion of contrast medium in an amount necessary for imaging of the lesion area and scanning the lesion area, and a process of stopping infusion of contrast medium once the time T1 or a time T1+α has elapsed from the start of contrast medium infusion.

### BACKGROUND OF THE INVENTION

1. Technical Field

The present invention relates to infusion methods for infusing contrast medium into a patient, and to lesion imaging systems that create a diagnostic image of a patient's lesion areas.

2. Description of the Related Art

To create diagnostic images of lesions, conventionally, a contrast medium is infused into a patient and then x-rays are irradiated onto the patient. The contrast medium in such cases is a pharmaceutical agent whose x-ray absorbency is different from that of human tissue, and for example is barium sulfate or an iodine solution. Of these, barium sulfate is administered orally, whereas iodine solutions are frequently used for injection or infusion into the body, but in the following description the contrast medium is not limited to iodine solutions.

FIG. 10 is a block diagram of a lesion imaging system that has been proposed in the past (for example, see JP H07-204176A). This system is for controlling the infusion of contrast medium into a patient 6 from outside an imaging room 4 into which the patient 6 has been led. Inside the imaging room 4 are provided an injector head 3, a head control unit 1, a first infrared transceiver 40, and an image capturing device 2, which is discussed later. Outside the imaging room 4 are arranged a second infrared transceiver 41 and a personal computer (hereinafter, “PC”) 5 that are in serial communication with the first infrared transceiver 40.

As shown in FIG. 11, two syringes A1 and B1 linked to a catheter 30 that is connected to the patient 6 are connected to the injector head 3. A penetration needle 31 is attached to the tip portion of the catheter 30. The one syringe A1 contains contrast medium and the other syringe B1 contains saline, and the injector head 3 is provided with a piston that pushes out the liquid within the syringes A1 and B1, and a piston drive actuator (not shown). The head control unit 1 receives signals from the first infrared transceiver 40 and controls the amount of piston movement and which of the syringes A1 or B1 to drive.

When performing a test, first the penetration needle 31 is stuck into the patient 6. The person performing the test on the patient 6, such as the examining technician, operates the PC 5 and inputs a signal for the infusion of contrast medium from the penetration needle 31. This signal is transferred to the head control unit 1 via the second and first infrared transceivers 41 and 40, and then, first contrast medium is infused into the patient 6 from the one syringe A1. After infusion is complete, saline is infused into the patient 6 from the other syringe B1 to infuse into the patient 6 any contrast medium remaining in the catheter 30. The contrast medium flows toward the lesion area through a vein or artery of the patient 6.

The patient 6 is then examined by the image capturing device 2. Generally the penetration needle 31 is removed, but it is also possible for it to be left in. The image capturing device 2 is a CT (computed tomography) device that as shown in FIG. 12 is provided with a table 21 that can move forward and backward, and on which the patient 6 is placed, and a circular opening 22 through which the table 21 passes. An x-ray tube 23 that irradiates x-rays onto the patient 6, and a detector 24 that receives x-rays that have passed through the patient 6 are provided in opposition to one another along the periphery of the opening 22, symmetrically flanking the center of the opening 22 between them, and the x-ray tube 23 and the detector 24 are provided such that they can rotate concentrically with respect to the center of the opening 22. The time Tscan that is required for infusion of a sufficient amount of contrast medium is found in advance from the extent of the lesion to be imaged, and that time Tscan is stored in the image capturing device 2 in advance.

The patient 6 into which the contrast medium has been infused lies down on the table 21. When the table 21 passes through the opening 22, the x-ray tube 23 and the detector 24 rotate concentrically with respect to the center of the opening 22, irradiating x-rays onto the lesion area for the time Tscan. As mentioned above, contrast medium has a different x-ray absorption then does body tissue, and thus when the contrast medium has arrived at the lesion, a cross-sectional image of the lesion of the patient 6 is obtained as a monochrome image. The monochrome image is displayed on a display 20 that is connected to the image capturing device 2. The task of irradiating x-rays from the x-ray tube 23 to obtain a sectional image of a lesion area is generally referred to as a “scan.”

Conventionally, a fixed amount of contrast medium has been infused into the patient 6, whoever that patient may be. Yet the time between infusion of the contrast medium and its arrival at the lesion area will vary, due for example to the location of the lesion area or the physical characteristics of the patient 6, and infusing a fixed amount of contrast medium regardless of the patient 6 has led to infusion of a greater amount of contrast medium than is actually necessary.

Because the contrast medium is foreign matter to the body, infusing a large amount of contrast medium into an allergy-prone patient 6, even through the contrast medium is subsequently excreted as urine, is undesirable. It is also known that administering a large amount to patients with reduced hepatic function or renal function, for example, may lead to the side effect of a worsening of their hepatic or renal functions.

### BRIEF SUMMARY OF THE INVENTION

It is an object of the present invention to reduce the amount of contrast medium that is infused into patients.

A method of infusing contrast medium of the present invention includes a process of infusing a small amount of contrast medium from the syringe A1 and using the image capturing device 2 to measure a time Tarv from a start of infusion until the contrast medium arrives at the lesion area,

a process of subtracting the arrival time Tarv from a time Tscan necessary for infusion of a sufficient amount of contrast medium, and taking this as a time T1,

a process of starting infusion of contrast medium in an amount necessary for imaging of the lesion area, and scanning the lesion area, and

a process of stopping infusion of contrast medium once the time T1 or a time T1+α has elapsed from the start of contrast medium infusion.

First, a small amount of contrast medium is infused from the syringe A1. When the contrast medium arrives at the lesion area, the image capturing device 2 shows the lesion area in white, for example. As shown in FIG. 5, a time Tarv from the start of infusion until the contrast medium has completely arrived at the lesion area, that is, until the lesion area appears whitest, is measured. The time Tscan required for the image capturing device 2 to image the lesion area is known in advance from the range of the lesion to be imaged, and thus the arrival time Tarv is subtracted from Tscan to obtain a time T1.

The lesion area is scanned at the same time that infusion of an amount of contrast medium necessary for imaging the lesion area is begun. The image capturing device 2 starts imaging the lesion area. After the time T1 has elapsed from the start of contrast medium infusion, then infusion of the contrast medium is stopped. Shown in a line diagram, the infusion of the contrast medium is stopped at the position of B in FIG. 6. The contrast medium arrives at the lesion after the time Tarv has elapsed from the start of infusion, and thus the last of the contrast medium arrives at the lesion area once the time of Tarv added to the time T1 has elapsed from the start of contrast medium infusion. The time obtained by summing the time T1 and the time Tarv is a time Tscan necessary for the image capturing device to infuse a necessary amount contrast medium, and thus only the amount of contrast medium necessary for the image capturing device 2 to image the lesion area is infused.

Consequently, it is possible to infuse an amount of contrast medium that corresponds to the position of the lesion or the physical characteristics of the patient, for example, allowing the amount of contrast medium that is infused to be reduced compared to conventional cases.

From the following detailed description in conjunction with the accompanying drawings, the foregoing and other objects, features, aspects and advantages of the present invention will become readily apparent to those skilled in the art.

### BRIEF DESCRIPTION OF THE SEVERAL VIEWS OF THE DRAWINGS

FIG. 1 is a block diagram of the lesion imaging system.

FIG. 2 is a block diagram of the head control unit interior.

FIG. 3 is a plan view showing the screen of the PC.

FIG. 4 is a graph showing infusion during the test bolus.

FIG. 5 is a graph plotting the relationship between the white level and time during the test bolus.

FIG. 6 is a graph plotting the relationship between contrast medium infusion time and the concentration of contrast medium that has arrived at the lesion area.

FIG. 7 is a graph plotting the relationship between contrast medium infusion time and the concentration of contrast medium that has arrived at the lesion area.

FIG. 8 is a flowchart showing the procedure during the test bolus.

FIG. 9 is a flowchart showing the procedure during the contrast medium infusion.

FIG. 10 is a block diagram of a conventional lesion imaging system.

FIG. 11 is a diagram showing the injector head.

FIG. 12 is a perspective view of the image capturing device.

### DETAILED DESCRIPTION OF THE INVENTION

### First Embodiment

An example of the present invention is described in detail below using the drawings. This example is characterized in being a method of infusing contrast medium.

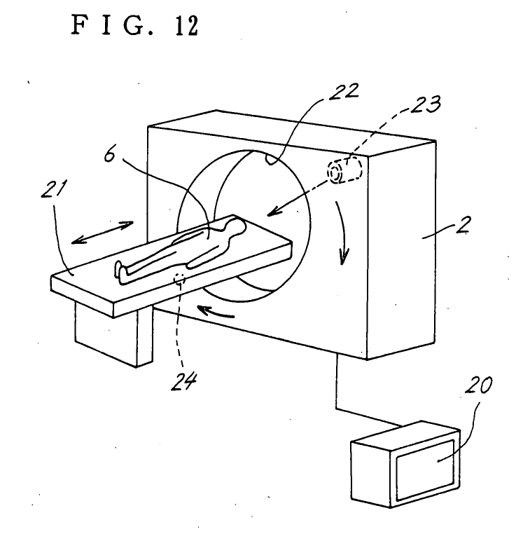

FIG. 1 is a block diagram of the lesion imaging system according to this example. An image capturing device 2, which is a CT device furnished with an injector head 3, a head control unit 1, a first infrared transceiver 40 and a display 20, is arranged inside an imaging room 4. A second infrared transceiver 41 and a PC 5 in serial communication with the first infrared transceiver 40 are arranged outside the imaging room 4. The PC 5 is provided with a screen 50 and an operation button 51, as is the case normally. The injector head 3 has the same structure as that of the conventional example depicted in FIG. 11.

Although the structure of this example resembles the conventional structure shown in FIG. 10, it is different in that the image capturing device 2 is linked to the head control unit 1. It should be noted that providing the first and second infrared transceivers 40 and 41 allows the person performing testing of the patient 6 to remotely operate the injector head 3. However, in place of this it is also possible to directly connect the PC 5 and the head control unit 1 to one another without providing the first and second infrared transceivers 40 and 41.

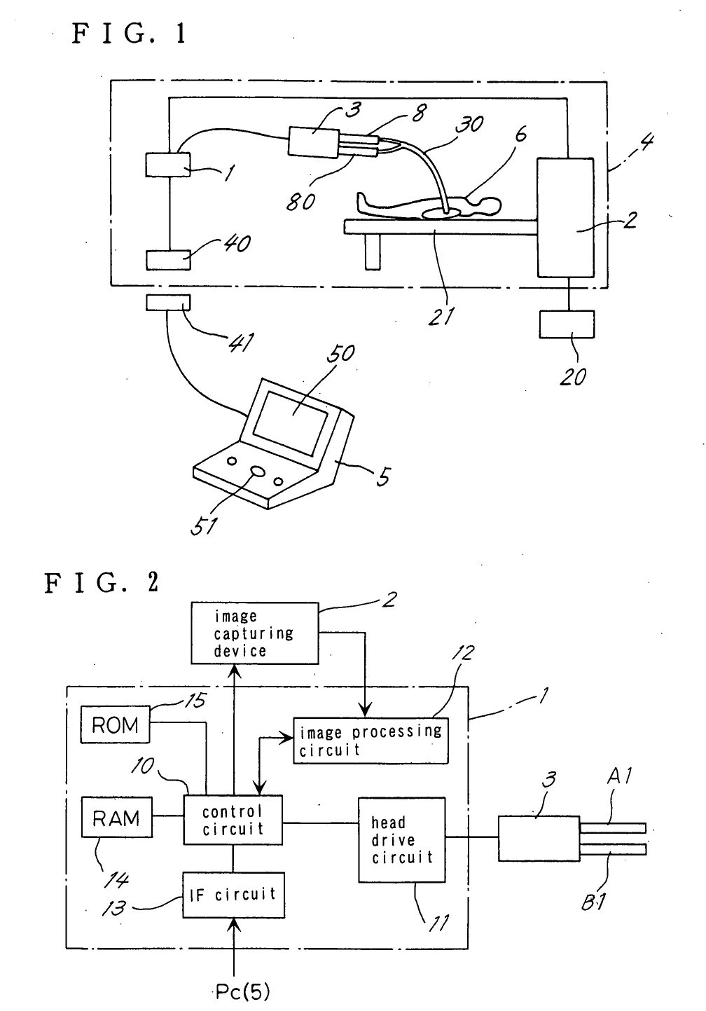

FIG. 2 is a block diagram of the internal structure of the head control unit 1. An operation signal from the PC 5 is input to a control circuit 10 via an interface (IF) circuit 13. The control circuit 10 is a microcomputer that has a timer function of dividing an internal operation clock. The control circuit 10 is connected to a ROM 15 storing the operation program, a head drive circuit 11 that is linked to the injector head 3, and to a RAM 14 that can store a contrast medium arrival time Tarv. The signal from the control circuit 10 is also sent to the image capturing device 2, and the brightness of the image from the image capturing device 2 is detected by an image processing circuit 12 and its white peak level is input to the control circuit 10.

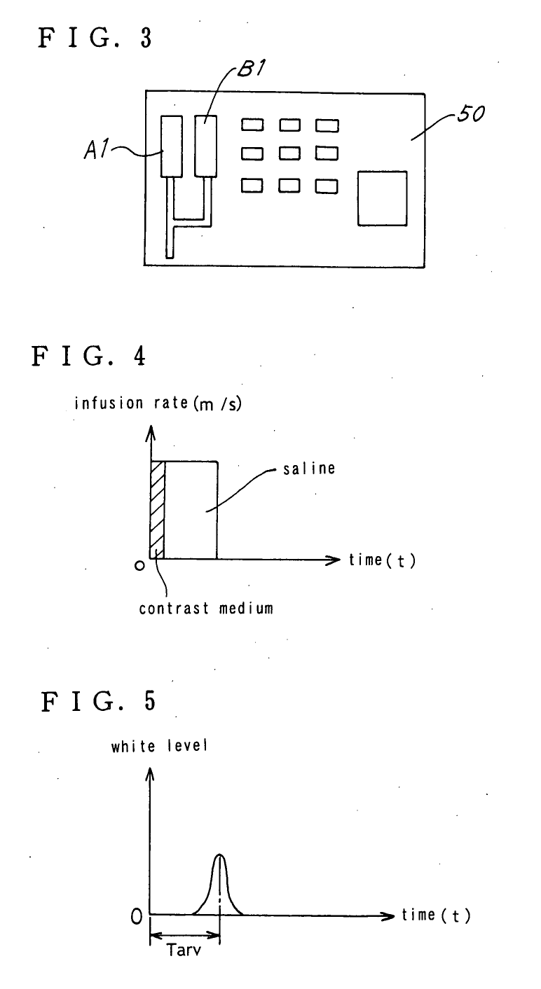

FIG. 3 is a plan view showing the screen 50 of the PC 5. An image of the syringes A1 and B1 that can be lit up is depicted on the screen 50, and the image of the syringe A1 lights up during contrast medium infusion and the image of the syringe B1 lights up during saline infusion. The screen 50 shows the infusion rate, the amount left to be infused, and the infusion time of the contrast medium or the saline. The person performing the testing operates the operation button 51 of the PC 5 to select a desired infusion rate, for example.

Method of Infusing Contrast Medium

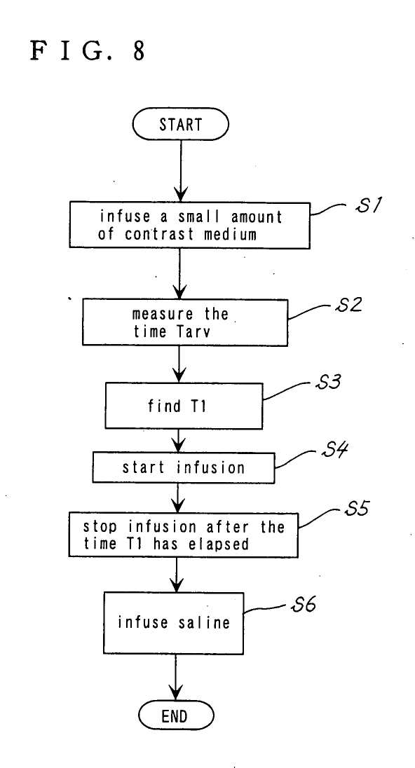

The method of infusing a contrast medium is described below using the graphs of FIG. 4 to FIG. 7 and the flowchart of FIG. 8. This example has the characteristic that first a small, detectable amount of contrast medium is infused.

The penetration needle 31 is stuck into the patient 6 and the patient 6 is led into the image capturing device 2. As the table 21 moves, the x-ray tube 23 emits x-rays into the lesion area of the patient 6. The person performing this testing of the patient 6 operates the PC 5 to start testing. A signal ordering the start of testing is delivered to the head control unit 1 via the second and first infrared transceivers 41 and 40, and the control circuit 10 receives this signal from the PC 5 and activates the head drive circuit 11. As shown in FIG. 4, a small amount of contrast medium on the order of 5 to 20 mL is infused into the patient 6 from the syringe A1 (S1). The infusion rate at this time is a velocity selected on the screen of the PC 5 and is on the order of about 10 mL per second, but this is not a limitation. After the contrast medium has been infused, then 15 to 30 mL of saline are infused from the syringe B1 to completely infuse the contrast medium remaining in the catheter 30.

When infusion of the small amount of contrast medium is started, the control circuit 10 activates an internal timer and transmits a signal to the image capturing device 2 to indicate that contrast medium has been infused. The image capturing device 2 successively displays the images obtained by scanning on the display 20 and transmits every unit image, for example, every frame, to the image processing circuit 12. Because the x-ray tube 23 is emitting x-rays into the lesion area, the lesion appears white when the contrast medium arrives at the lesion. As shown in FIG. 5, when the white level of the received images has reached a peak, the image processing circuit 12 transmits a signal to the control circuit 10 to indicate that the contrast medium has completely arrived at the lesion area. Whether or not the white level has peaked is determined using the well-known method of comparing the white level of the image that is received immediately prior and white level of the currently received image.

The control circuit 10 measures the time Tarv from the start of infusion until the contrast medium has completely arrived at the lesion area (S2), and then temporarily stores the value of this time Tarv in the RAM 14. It is also possible to send the value of the time Tarv to the PC 5 and display it on the screen 50.

The control circuit 10 then subtracts the value of the time Tarv in the RAM 14 from the time Tscan necessary for imaging and takes the result as T1 (S3). The control circuit 10 stores the value of T1 in the RAM 14. The time Tscan necessary to infuse a sufficient amount of contrast medium is stored in the image capturing device 2 in advance, and it is also possible to store it in the ROM 15 in advance, even though the control circuit 10 reads it from the image capturing device 2. The operation of infusing a small amount of contrast medium until the time T1 is found is referred to as a “test bolus,” with “bolus” being a medical term that means a single infusion.

After the test bolus is finished, the control circuit 10 actuates the head drive circuit 11 to infuse into the patient 6 an amount of contrast medium that is necessary for testing of the lesion area from the syringe A1 (S4). The control circuit 10 activates its timer function. The image capturing device 2 starts imaging and each frame transmits to the image processing circuit 12.

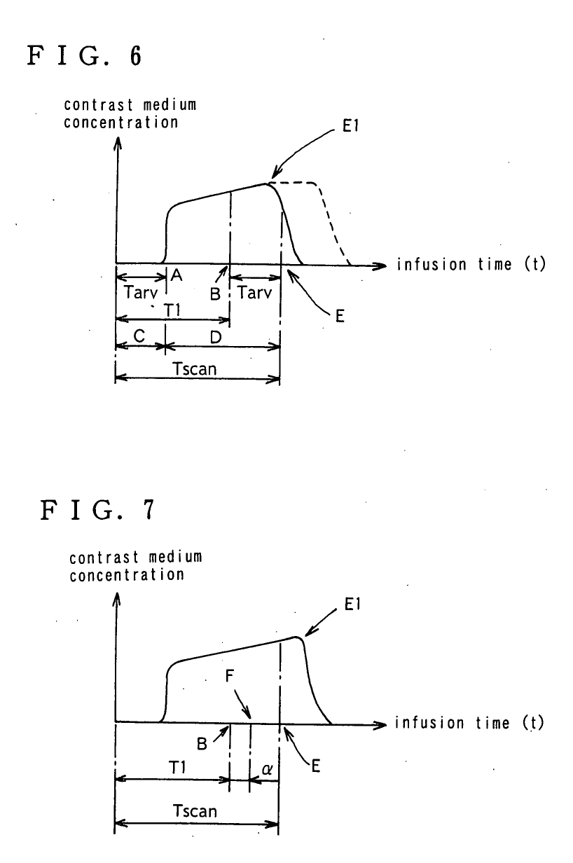

FIG. 6 is a graph plotting the contrast medium concentration in the lesion area versus infusion time. The concentration of the contrast medium in the lesion area is proportional to the white level of the captured image, and is found from the white level. Point A in FIG. 6 shows the time Tarv at which the contrast medium arrives at the lesion area, and from the point A the white level begins to rise, that is, the contrast medium begins to arrive at the lesion. Point E is the point at which the time Tscan required for a sufficient amount of contrast medium to be infused has elapsed since the start of scanning, and the point E1 immediately before point E is where the white level peaks. It should be noted that the time Tscan required for a sufficient amount of contrast medium to be infused from the start of scanning and the point at which the white level peaks do not always match, and generally one is either before or after the other. A case in which the white level peaks at a point beyond the point E is discussed later.

When infusion of the contrast medium is begun, the image capturing device 2 rotates the x-ray tube and the detector 24 to perform a scan. However, because it is known that the contrast medium does not arrive at the lesion area until point A after the start of scanning, it is difficult to obtain an image of the lesion area even if x-rays are emitted from the x-ray tube 23. Accordingly, if during the period C from the start of scanning until the point A the x-ray tube 23 is rotated and intermittently emits low-power level x-rays, and during the period D from the point A to the point E it continually emits powerful x-rays for imaging, then the amount of x-ray radiation to the patient 6 can be reduced.

The control circuit 10 reads the value of the time Tarv from the RAM 14, and at the point B after the time T1 has elapsed from the start of contrast medium infusion, it actuates the head drive circuit 11 to stop infusion of the contrast medium (S5). The contrast medium arrives at the lesion area once the time Tarv has elapsed from infusion, and thus the last of the contrast medium arrives at the lesion area after the time Tarv has elapsed from point B. After infusion of the contrast medium is stopped, saline is infused in order to flush any contrast medium remaining in the catheter 30 into the patient 6 (S6).

In other words, the last of the contrast medium arrives at the lesion after the time obtained by summing the time Tarv and the time T1 has elapsed from the start of infusion of the contrast medium. The time obtained by summing the time Tarv and the time T1 is the time Tscan required for the image capturing device 2 to infuse a sufficient amount of contrast medium, and thus the image capturing device 2 infuses only the amount of contrast medium required for imaging the lesion.

The image capturing device 2 displays the images obtained by scanning on the display 20. A physician can look at images of the lesion shown on the display 20 and make a diagnosis.

Conventionally, a fixed amount of contrast medium was infused irrespective of the patient 6, and this fixed amount was a sufficient amount equal to or greater than that required for scanning. Consequently, depending on the patient 6, there was the possibility that too much contrast medium would be infused, in which case the relationship between the contrast medium infusion time and the concentration of the contrast medium in the lesion is shown by the dotted line in FIG. 6.

In this example, a test bolus is used to determine the time Tarv for the contrast medium to arrive at the lesion, the contrast medium is then infused, and then infusion is stopped once a time T1 has elapsed. Consequently, it is possible to infuse an amount of contrast medium that corresponds to the position of the lesion or the physical characteristics of the patient 6, allowing the amount of contrast medium that is infused to be reduced compared to what was the case conventionally.

It should be noted that as mentioned above, the time Tscan necessary for infusing a sufficient amount of contrast medium from the start of scanning and the point where the white level peaks do not always match, and generally one falls before or after the other. Consequently, as shown in FIG. 7, there are instances where the white level reaches a peak at a point E1 beyond the point E, which is the point that the time Tscan necessary for infusion of a sufficient amount of contrast medium has elapsed from the start of scanning.

In this case, the infusion of contrast medium can be stopped after a time α has further passed beyond the point B at which the time T1 has elapsed from the start of infusion of the contrast medium. As regards stopping the infusion of contrast medium after the passage of a time α from the time T1, it is conceivable that the person performing testing could manually input the time α through the PC 5 for example in a case where a clear image was not obtained when scanning the lesion area and scanning of the same lesion area is to be performed a second time. In this case, the control circuit 10 stops infusion after the time T1+α has elapsed from the start of infusion. The applicant of the present invention has envisioned the time α being about one second, but this depends on the rate of infusion of the contrast medium, and there is no limitation to one second.

### Second Embodiment

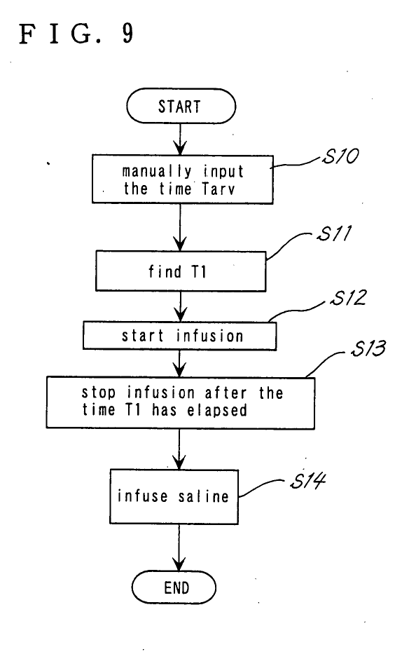

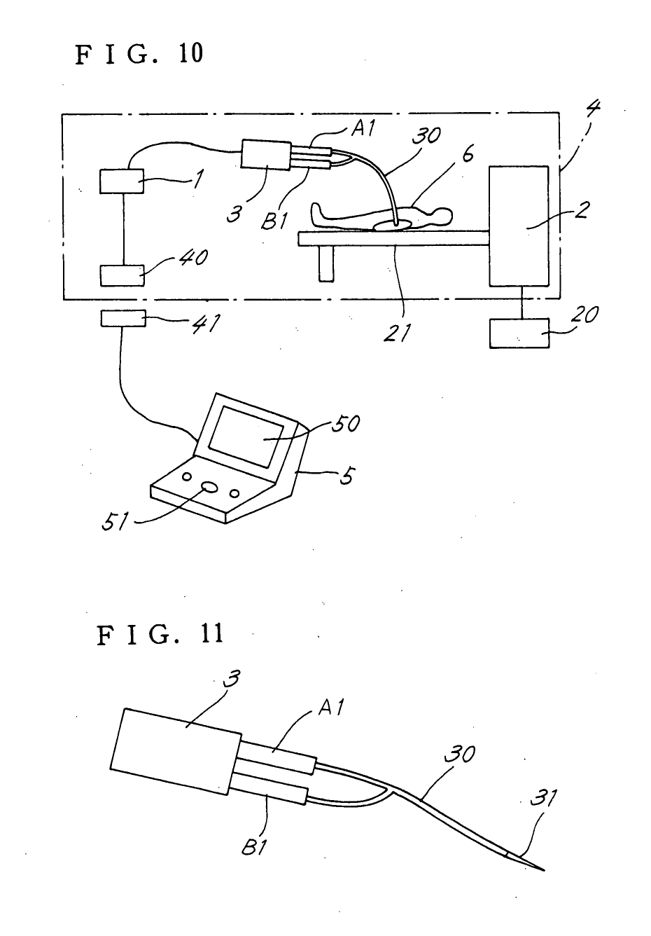

In a case where the same lesion area of the same patient 6 is to be tested repeatedly, it is a nuisance to each time perform a test bolus to find the time Tarv. Also, the time Tarv is substantially constant for the same lesion area of the same patient 6. Accordingly, in such a case the person performing testing can manually input the time Tarv and forego the test bolus. This operation is shown in the flowchart of FIG. 9.

The person performing the testing inputs to the PC 5 that he is foregoing the test bolus. The PC 5 sends this signal to the head control unit 1, and the control circuit 10 calls up from the ROM 15 a program from which the test bolus has been obviated.

The person performing testing manually inputs the time Tarv (S10). The value of the time Tarv that is used is, as discussed above, the value displayed on the screen 50 of the PC 5 when the testing was performed initially. The control circuit 10 subtracts Tarv from the value for Tscan to find T1 (S11). Then, infusion is stopped once the time T1 has elapsed from the start of infusion of the contrast medium (S12, S13), saline is infused (S14), and an image of the lesion is displayed on the display 20.

In the above discussion, a CT device served as an illustrative example of the image capturing device 2, but in place of this it is also possible to use a MRI (magnetic resonance imaging) device. It is well known that instead of emitting x-rays, an MRI device emits a powerful magnetic field and electromagnetic waves to obtain a magnetic resonance image.

In a case where a MRI device is used, the applicant of the present invention envisions the infusion of 1 to 3 mL of contrast medium at the time of the test bolus, but there is no limitation to this value.

When the contrast medium concentration is low at the time of the test bolus, then there is a risk that a clear image will not be obtained even if the amount of contrast medium necessary to image the lesion area is infused. At such a time, the concentration of the contrast medium can be increased when infusing the amount of contrast medium necessary to image the lesion area is infused.

Only selected embodiments have been chosen to illustrate the present invention. To those skilled in the art, however, it will be apparent from the foregoing disclosure that various changes and modifications can be made herein without departing from the scope of the invention as defined in the appended claims. Furthermore, the foregoing description of the embodiments according to the present invention is provided for illustration only, and not for limiting the invention as defined by the appended claims and their equivalents.

What is claimed is:

1. A contrast-medium infusion method using a system furnished with a syringe through which contrast medium is introduced, a control unit for controlling contrast-medium infusion through the syringe, and an image capturing device for scanning a lesion area in a patient into whom contrast medium has been infused through the syringe, and that pre-stores a time T_{scan} necessary for infusion of a quantity of contrast medium sufficient to image the lesion area, the method comprising:
a step of infusing a small amount of contrast medium from the syringe and, using the image capturing device, of measuring time Tₐᵣᵥ from start of infusion until the contrast medium arrives at the lesion area;
a step of subtracting the arrival time Tₐᵣᵥ from the time T_{scan}, and letting the result be time T₁;
a step, together with starting of contrast-medium infusion in the amount necessary to image the lesion area, of scanning the lesion area; and
a step of stopping infusion of the contrast medium once the time T₁ or a time T₁+α (wherein α is a positive number) has elapsed from the start of contrast medium infusion.

2. The contrast-medium infusion method according to claim 1, further comprising a step of infusing saline after the infusion of contrast medium has been stopped.

3. The contrast-medium infusion method according to claim 1 or 2, wherein the intensity of x-rays or magnetic flux irradiated onto the patient during scanning is lowered from the start of contrast medium infusion to the arrival time Tₐᵣᵥ, and after the arrival time Tₐᵣᵥ has elapsed, the irradiated x-ray or magnetic-flux intensity is raised.

4. A contrast-medium infusion method using a system furnished with a syringe through which contrast medium is introduced, a control unit for controlling contrast-medium infusion through the syringe, and an image capturing device for scanning a lesion area in a patient into whom contrast medium has been infused through the syringe, the method comprising:
a step of an examining person inputting a time Tₐᵣᵥ from start of contrast medium infusion until the contrast medium arrives at the lesion area;
a step of subtracting the arrival time Tₐᵣᵥ from a time T_{scan} necessary for infusion of a quantity of contrast medium sufficient to image the lesion area, and letting the result be time T₁;
a step, together with starting of contrast-medium infusion in the amount necessary to image the lesion area, of scanning the lesion area; and
a step of stopping infusion of the contrast medium once the time T₁ or a time T₁+α has elapsed from the start of contrast medium infusion.

5. A lesion imaging system comprising a syringe through which contrast medium is introduced, a control unit for controlling contrast-medium infusion through the syringe, and an image capturing device for scanning a lesion area in a patient into whom contrast medium has been infused through the syringe, wherein a time T_{scan} necessary for infusion of a quantity of contrast medium sufficient to image the lesion area is pre-stored in the image capturing device, and wherein the image capturing device is linked to a display for displaying an image of the lesion area that has been imaged, wherein the control unit stores a program for:
infusing a small amount of contrast medium from the syringe and, using the image capturing device, of measuring time Tₐᵣᵥ from start of infusion until the contrast medium arrives at the lesion area;
subtracting the arrival time Tₐᵣᵥ from the time T_{scan} to obtain a time T₁;
together with starting of contrast-medium infusion in the amount necessary to image the lesion area, scanning the lesion area; and
stopping infusion of the contrast medium once the time T₁ or a time T₁+α has elapsed from the start of contrast medium infusion.
